(19) United States
(12) Patent Application Publication
(10) Pub. No.: US 2006/0100290 A1
(43) Pub. Date: May 11, 2006

(54) Treatment of allergic rhinitis and asthma

(76) Inventor: Leslie Joe Dunaway, Louisville, KY (US)

Correspondence Address: SIEBERTH & PATTY, LLC, 4703 BLUEBONNET BLVD, BATON ROUGE, LA, 70809, US

(21) Appl. No.: 10/532,439

(22) PCT Filed: Jul. 27, 2004
(86) PCT No.: PCT/US04/24125

§ 371 Date: Apr. 22, 2005

Related U.S. Application Data
(60) Provisional application No. 60/490,644, filed on Jul. 28, 2003.

(51) Int. Cl. A61K 31/138

(52) U.S. Cl. 514/651

(57) ABSTRACT

This invention provides a method of treating allergic rhinitis in a mammal comprising administering a therapeutically effective amount of atomoxetine to a mammal in need of such treatment, and also provides a method of treating asthma in a mammal comprising administering a therapeutically effective amount of atomoxetine to a mammal in need of such treatment.

### REFERENCE TO RELATED APPLICATION

This Application claims the benefit of prior co-pending U.S. Provisional Application No. 60/490,644 filed on Jul. 28, 2003, the disclosure of which is incorporated herein by reference.

### TECHNICAL FIELD

This invention relates to the treatment of allergic rhinitis and asthma.

### BACKGROUND

Atomoxetine, sometimes also called tomoxetine, is a selective norepinephrine reuptake inhibitor. One chemical designation for atomoxetine is (−)-N-Methyl-3-phenyl-3-(o-tolyloxy)-propylamine. For teachings relating to this composition, see, e.g., U.S. Pat. Nos. 4,229,449 and 4,271,160, which are hereby incorporated by reference. Syntheses of atomoxetin are described in U.S. Pat. Nos. 4,018,895, 4,194,909, 4,314,081, 4,777,291, and 6,541,668, which are hereby incorporated by reference. Also in this connection, see U.S. Pat. No. 6,008,412, regarding processes for resolving N-methyl-3 (R,S)-hydroxy-3 -phenylpropylamine.

### SUMMARY OF INVENTION

However, it has now been found that atomoxetine is useful in the treatment of allergic rhinitis and/or asthma The precise mechanism by which atomoxetine produces its therapeutic effects in allergic rhinitis and asthma is unknown, but, without wishing to be bound by theory, is thought to be related to inhibiting reuptake of norepinephrine in an area of the brain responsible for regulation of the reactivity of the airways of the upper respiratory and lower respiratory tracts through stabilization of the epithelial membrane which lines these tracts to curb the transport of allergens across this membrane. It is a well known fact that allergic rhinitis and asthma are associated with an increased reactivity of the airways on exposure to environmental allergens. Thus reduction of the airway reactivity with a therapeutic dose of atomoxetine will decrease or even totally eliminate the symptoms of allergic rhinitis and asthma.

One embodiment of this invention is a method of treating allergic rhinitis in a mammal comprising administering a therapeutically effective amount of atomoxetine to a mammal in need of such treatment. Other embodiments of the invention include a method of treating asthma in a mammal comprising administering a therapeutically effective amount of atomoxetine to a mammal in need of such treatment, and a method of treating allergic rhinitis and asthma in a mammal comprising administering a therapeutically effective amount of atomoxetine to a mammal in need of such treatment.

This and other embodiments and features of this invention will be still further apparent from the ensuing description and appended claims.

### FURTHER DETAILED DESCRIPTION OF THE INVENTION

The compound relating to this invention is atomoxetine, which is a well-known drug, the chemical name of which is (R)-(−)-N-methyl-3-(2-methylphenoxy)-3-phenylpropylamine. It is regularly used as a salt, and pharmaceutically acceptable salts are-included in the term atomoxetine as used here. As used herein, the term “pharmaceutically acceptable salt” refers to those salts which are, within the scope of sound medical judgment, suitable for use in contact with the tissues of humans and other mammals without undue toxicity, irritation, allergic response and the like, and are commensurate with a reasonable benefit/risk ratio. Pharmaceutically acceptable salts are well known in the art. The HCl salt of atomoxetine is an especially preferred pharmaceutically acceptable salt of atomoxetine in the practice of this invention. The HCl salt of atomoxetine has the chemical formula:

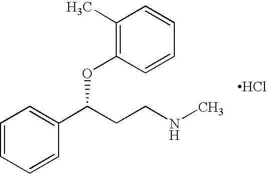

Allergic rhinitis is characterized by any combination of the symptoms of sneezing, clear nasal discharge, nasal congestion, clear postnasal drainage, and itchy, watery eyes occurring in a temporal relationship to allergen exposure. Following allergen exposure, the allergen is bound by Immunoglobulin E (IgE) molecules attached to a mast cell, which causes the release from the mast cell of many pharmacoactive compounds which lead to the production of allergy symptoms. Asthma is a disease of airways that is characterized by increased responsiveness of the tracheobronchial tree to a multiplicity of stimuli. Asthma is manifested physiologically by a widespread narrowing of the airways, and clinically by paroxysms of dyspnea, cough, and wheezing. As in allergic rhinitis, symptoms are initiated by binding of allergens to IgE attached to mast cells, causing the release from mast cells of many pharmacoactive compounds.

The effective dose of atomoxetine for the treatment of allergic rhinitis and/or asthma for humans is in the range from about 5 mg/day to about 100 mg/day. The preferred adult dose is in a range from about 10 mg/day to about 100 mg/day. A more preferred adult dose is in the range from about 10 to about 80 mg/day. The preferred children's dose is in a range from about 5 mg/day to 60 mg/day, more preferably from about 10 to about 60 mg/day. Typically, the dose of atomoxetine is about 1.2 to about 1.4 mg of atomoxetine per kilogram of weight, but the optimum dose for each patient, as always, must be determined by the attending physician, taking into account other medications which the patient requires, severity of the illness, and all other pertinent patient history, as well as the patient's weight.

Since atomoxetine is readily orally absorbed and requires only once/day administration, oral administration is highly preferred. Other methods of administration may be used when necessary, or when oral administration is inconvenient. Atomoxetine may be produced in the form of a clean, stable crystal, and thus is easily formulated in the usual oral pharmaceutical forms, such as tablets, capsules, and suspensions.

The method of the present invention is effective in the treatment of both children and adults who suffer from symptoms of allergic rhinitis and/or asthma. Examples 1-4 demonstrate the effectiveness of atomoxetine in treating the symptoms of allergic rhinitis and asthma in humans.

The following examples are presented for purposes of illustration, and are not intended to impose limitations on the scope of this invention.

### EXAMPLE 1

An 11 year old female with an 8 year history of allergic rhinitis and a 3 year history of moderate persistent asthma was given a 60 mg daily dose of atomoxetine. Within three weeks of initiation of treatment with atomoxetine, she was able to wean all traditional allergic rhinitis and asthma treatments (antihistamines, leukotriene modifiers, bronchodilators, and inhaled corticosteroids). During the following year, she was monitored while on atomoxetine mono therapy, and remained symptom free during the entire period of monitoring.

### EXAMPLE 2

A 51 year old with a 24 year history of severe allergic rhinitis, immunotherapy failure, and a 10 year history of moderate persistent asthma was given a 100 mg daily dose of atomoxetine. Within 10 days he was able to wean all traditional allergic rhinitis and asthma treatments (antihistamines, nasal corticosteroids, bronchodilators, and inhaled corticosteroids).

During the following 10 months, he was monitored while on atomoxetine mono therapy, and remained symptom free during the entire period of monitoring.

### EXAMPLE 3

A 25 year old female with a 20 year history of allergic rhinitis was given a80 mg daily dose of atomoxetine. Within 7 days, she was able to wean all traditional allergic rhinitis (antihistamines and nasal corticosteroids). She has remained symptom free for at least nine months with atomoxetine mono therapy.

### EXAMPLE 4

A 40 year old female with a 10 year history of allergic rhinitis and a 5 year history of moderate persistent asthma was given a 80 mg daily dose of atomoxetine. Within 10 days, she was able to wean all traditional allergic rhinitis and asthma treatments (antihistamines, nasal corticosteroids, bronchodilators, theophylline, inhaled corticosteroids, and leukotriene modifiers). Her last pulmonary function test while on traditional treatments showed an FEV 1 of 68% and repeat pulmonary function test after 6 weeks of atomoxetine mono therapy showed an FEV1 percent of 93%. She has remained symptom free for at least 7 months with atomoxetine mono therapy.

Here, as is known in the art, FEV1 percent is the ratio of FEV1 to the predicted FEV1, which is obtained from standardized tables based on age, weight, height, and sex; FEV1 is the volume of air that is forcefully exhaled from the lungs in one second.

This invention is susceptible to considerable variation within the spirit and scope of the appended claims.

1. A method of treating allergic rhinitis in a mammal comprising administering a therapeutically effective amount of atomoxetine to a mammal in need of such treatment.

2. A method according to claim 1 wherein the atomoxetine is the HCl salt of atomoxetine.

3. A method according to claim 1 wherein the mammal is human.

4. A method according to claim 1 wherein the administering is by oral administration.

5. A method according to claim 1 wherein the atomoxetine is the HCl salt of atomoxetine, and wherein the mammal is human.

6. A method according to claim 1 wherein the atomoxetine is the HCl salt of atomoxetine, and wherein the administering is by oral administration.

7. A method according to claim 1 wherein the atomoxetine is the HCl salt of atomoxetine, wherein the mammal is human, and wherein the administering is by oral administration.

8. A method of treating asthma in a mammal comprising administering a therapeutically effective amount of atomoxetine to a mammal in need of such treatment.

9. A method according to claim 8 wherein the atomoxetine is the HCl salt of atomoxetine.

10. A method according to claim 8 wherein the mammal is human.

11. A method according to claim 8 wherein the administering is by oral administration.

12. A method according to claim 8 wherein the atomoxetine is the HCl salt of atomoxetine, and wherein the mammal is human.

13. A method according to claim 8 wherein the atomoxetine is the HCl salt of atomoxetine, and wherein the administering is by oral administration.

14. A method according to claim 8 wherein the atomoxetine is the HCl salt of atomoxetine, wherein the mammal is human, and wherein the administering is by oral administration.

15. A method of treating allergic rhinitis and asthma in a mammal comprising administering a therapeutically effective amount of atomoxetine to a mammal in need of such treatment.

16. A method according to claim 15 wherein the atomoxetine is the HCl salt of atomoxetine.

17. A method according to claim 15 wherein the mammal is human.

18. A method according to claim 15 wherein the administering is by oral administration.

19. A method according to claim 15 wherein the atomoxetine is the HCl salt of atomoxetine, and wherein the mammal is human.

20. A method according to claim 15 wherein the atomoxetine is the HCl salt of atomoxetine, and wherein the administering is by oral administration.

21. A method according to claim 15 wherein the atomoxetine is the HCl salt of atomoxetine, wherein the mammal is human, and wherein the administering is by oral administration.
